(19) United States
(12) Patent Application Publication
(10) Pub. No.: US 2009/0054350 A1
(43) Pub. Date: Feb. 26, 2009

(54) PROCESS FOR THE STERILE CONCENTRATION OF COLLAGEN PREPARATIONS, AND COLLAGEN PREPARATIONS OBTAINED

(76) Inventors: Jean-Louis Tayot, LaTour De Salvagny (FR); Florent Sapin, Lyon (FR)

Correspondence Address: STITES & HARBISON PLLC, 1199 NORTH FAIRFAX STREET, SUITE 900, ALEXANDRIA, VA, 22314, US

(21) Appl. No.: 12/194,100

(22) Filed: Aug. 19, 2008

Related U.S. Application Data
(60) Provisional application No. 60/957,562, filed on Aug. 23, 2007.

(51) Int. Cl. A61K 38/39

(52) U.S. Cl. 514/21

(57) ABSTRACT

The invention relates to a process for the preparation of sterile collagen, especially injectable collagen, comprising the steps of preparing a purified acidic collagen solution, filtering that solution through a filter or a sterile membrane, precipitating the filtered solution in the form of a suspension, and concentrating the collagen suspension, in which process the suspension is placed in an elongate supple tubular filter constituting a porous filtering wall, permeable to the liquid but not permeable to the collagen fibres, and is concentrated by the slow passage of the liquid through the wall, without any pressure other than gravity, in order to prevent the filtering wall from becoming clogged by the collagen fibres, optionally with absorption of the liquid by an absorbent layer covering the wall or forming part of that wall, and the subsequent flowing-away and/or evaporation of the liquid via the face of the layer remote from the wall, and collagen preparations obtained.

The present invention relates to a process for the sterile concentration of preparations of collagens, especially collagens of type I, II, III, IV, V, or other types referred to as minor collagens, or their mixtures, for example of type I+III; these collagens may be of animal origin, for example bovine or porcine origin, or human origin, in which case they are preferably of placental or dermal origin, the term collagen naturally including collagens obtained by synthesis, for example by expression in recombinant organisms.

Collagen preparations, in various injectable or implantable forms, are well known, as described, for example, by U.S. Pat. Nos. 3,949,073 in the name of Daniels and Knapp and 4,488,911 in the name of Luck and Daniels, who are the founders of the Collagen Corporation in California and the creators of the bovine collagen implants “Zyderm®”. The well-known difficulty encountered in preparing these injectable implants is to effect their concentration under strictly sterile conditions.

The concentration of acidic collagen solutions in dialysis tubing is described in U.S. Pat. No. 3,949,073, by absorption of water by means of an absorbent fabric placed around a dialysis tube. However, this technique does not permit the preparation of a directly injectable physiological product. The neutralisation of the acidic gel by dialysis against a physiological solution is not envisaged, probably owing to the time necessary to dialyse a very viscous solution of concentrated collagen and the difficulty involved. For example, when a concentrated collagen solution is subjected to dialysis, the very high oncotic pressure of the collagen gel rapidly brings about an influx of liquid into the tubing which may burst. In addition, this influx of liquid dilutes the collagen and reduces the concentration thereof, rendering this technique ineffectual. The same patent mentions that the fibrillar collagen suspensions are concentrated by centrifugation.

In U.S. Pat. No. 4,488,911, the two extracts below give a state of the art in the 1980s which is still practised today:

“In one simplified technique the collagen in solution is dialysed against a dilute carboxylic acid solution ( . . . ) at ambient temperature, then dialysed with rotational shear against dilute alkaline phosphate e.g. 0.02 M disodium phosphate for a sufficient period of time, until micropolymers form. Usually 2 to 8 hours (preferably about 4 to 6 hours) at ambient temperatures are required, although temperatures of about 15° C. to 40° C. can be used. The NFMs which form are collected by centrifugation, washed with water and stored. The ranges set forth previously are applicable to this procedure.

After removal of the dialysis bag from the solution, the fibrous micropolymers are collected by centrifugation. They may then be used directly, or stored wet at 4° C., or alternatively, freeze-dried for indefinite storage.”

However, the collection of the collagen fibres and their concentration by centrifugation pose the major problem of carrying out these operations without the risk of introducing contaminating infectious agents and thus impairing the contents of the syringes without any guarantee of bacterial and fungal sterility. For, centrifugation assumes that the collagen suspension is distributed in previously sterilised centrifugation cups, that the cups are introduced into a non-sterile machine, generally outside the sterile area, and that they are opened at the end of the centrifugation process, without reintroducing microbes into the product or the sterile area protecting it. Validation of the sterile conditions in such a process is very difficult and the necessary manual operations are lengthy, very tricky, risky and expensive. Assurance of the final sterility of the injectable product is thus reduced and this may lead to some local infection and intolerance reactions after injection.

U.S. Pat. No. 5,002,071 in the name of Harrell provides a solution to the problem of sterility by subjecting the injectable collagen to a final sterilisation by irradiation. However, apart from the risks of adversely affecting the collagen, this technique does not provide a solution for the physical elimination of the microbes introduced during the manual operations. Although these microbes are killed by the irradiation, the absence of bacterial bodies is not guaranteed and therefore pyrogenic reactions to the injection of these products cannot be excluded.

The present invention relates, in particular but not exclusively, to injectable preparations, in particular for filling tissue, an important application of which consists in the filling of cutaneous wrinkles.

Processes for the preparation of collagen from animal or human tissue, for example the human placenta, are already known. These processes are described, for example, in the above three documents and many others mentioned, in particular, in U.S. Pat. No. 5,436,135 in the name of Tayot and Tardy.

The usual steps consist in operations for washing the tissue to be treated, its partial digestion by proteases, such as pepsin or trypsin, and the extraction and purification of the acid-soluble collagen by saline precipitations. The collagen obtained, for example in precipitated form or in the form of a dried powder, is then dissolved in an acidic solution, for example in order to undergo sterilisation by filtration through a sterilising membrane. This solubilisation is generally effected at a low concentration of from 1 to 3 mg/ml to obtain a reduced viscosity permitting sterilising filtration. As indicated by the prior art already mentioned, the preparation of sterile and injectable concentrated fibrillar suspensions subsequently involves a reprecipitation of the collagen forming an extremely dilute suspension. This suspension is very difficult to filter or to concentrate under strict aseptic conditions in order to obtain a sterile injectable suspension at a concentration of the order of from 30 to 60 mg/ml (that is to say, from 3 to 6%) because collagen in suspension very rapidly clogs the filters and in any case it is very difficult to recover the paste from the usual filters. Therefore, as discussed above, this concentration is generally effected by centrifugation which, bearing in mind the large volumes to be concentrated, means lengthy manual operations which are very tricky and which in any case involve the risk of losing sterility when they are performed outside a sterile zone.

The present invention proposes to overcome these disadvantages and to provide a simple and rapid process of concentration which does not involve transfer between various installations, so that the concentration step, as well as the final distribution in sterile receptacles can be carried out in the same sterile environment, for example in a sterile container or beneath a sterile hood with vertical or horizontal laminar flow.

The invention relates to a process for the preparation of sterile collagen, especially injectable collagen, comprising the steps of preparing a purified acidic collagen solution, sterilising that solution, especially by filtration through a filter or a sterilising membrane, precipitating the sterile solution in the form of a suspension and concentrating the collagen suspension, in which process the suspension is placed in an elongate supple tubular filter constituting a porous filtering wall, permeable to the liquid but not permeable to the collagen fibres, and is concentrated by the slow passage of the liquid through the wall, without any pressure other than gravity, in order to prevent the filtering wall from becoming clogged by the collagen fibres.

The passage of the liquid can be increased, especially at the end of the concentration process, by absorption of the liquid by a previously sterilised absorbent layer applied to the wall, or forming part of that wall. In that case, the liquid is released outside the absorbent layer by flowing out of the absorbent layer when it is saturated, and/or by evaporating on coming into contact with a stream of sterile air.

In an especially preferred embodiment of the invention the steps are carried out in the same sterile environment, in which it is preferably also possible to carry out the preliminary steps of sterile filtration of the purified collagen and/or the subsequent steps of distributing the concentrated collagen in sterile syringes or receptacles; for the small space requirement of the means of concentration according to the invention enables all of these operations to be carried out, for example, in the same sterile container or sterile hood with a flow of sterile air, by means of an automatic device, under the control of an operator at the beginning and end of the concentration process.

Preferably, the permeable wall is constituted by a simple, supple, tubular filtering sheet which can be closed off at its lower end and which may contain the collagen suspension. At its opposite face, the sheet may be in contact with the absorbent layer, for example a sheet of cellulose or absorbent paper which has been sterilised beforehand by irradiation.

By lengthening such a tube to a sufficient extent, and by connecting it to the sterile reservoir containing the suspension to be concentrated, it is surprisingly possible to obtain progressive concentration of a continuous flow of suspension.

The porosity, or the mesh in the case of a woven textile, must be such that the aqueous liquid can pass easily through the wall, while the collagen tends to clog it. The moisture in the wall escapes spontaneously by sweating, in the absence of any pressure other than simple gravity. The finer the collagen precipitate, the more greatly the porosity of the filtering sheet or wall must be reduced. For the finest precipitates, the latter may be, for example, a woven textile whose threads are very close together and provide a porosity estimated by the manufacturer to be 1 micron. For the most structured precipitates or precipitates having broad fibrils, a porosity of as much as approximately 100 microns can be used. The liquid can also be dealt with by the absorbent layer, if present, for example at the end of the concentration process, and arrives at the external wall thereof, from where it flows away or evaporates. The material forming the absorbent layer must be such that it absorbs this moisture but without retaining it permanently, so that when it is saturated it readily releases this moisture at its opposite face remote from the wall permeable to the aqueous liquid of the suspension.

The absorbent layer may be, for example, a normal filter paper which is made of cellulose or other hydrophilic polymers of the polyester type or the like, in one or more thicknesses, and which has been sterilised beforehand by beta or gamma irradiation.

The device implementing the invention may comprise a means, receptacle or duct for receiving the liquid flowing away, or also a ventilation device for evaporating the liquid at the external wall of the absorbent layer. The creation of a partial vacuum around the external wall at the end of the concentration process may also be provided for.

The invention relates also to the resulting sterile concentrated preparations of collagen and especially injectable collagen, for example, for filling tissue, for example cutaneous wrinkles.

Instead of an injectable preparation, it is, however, also possible to continue to treat the collagen, starting from the concentrated suspension obtained, in order to prepare other implantable collagen materials that are solid or non-solid, and sterile or sterilisable.

Other advantages and features of the invention will emerge on reading the following description which is given by way of non-limiting example.

### EXAMPLE 1

### Concentration of a Suspension of Neutral Fibrillar Collagen Precipitate

An acidic solution of collagen type I or I+III, having a pH of from 2 to 3, is prepared by dissolving a lyophilised or acetone powder of collagen fibres in 0.02N hydrochloric acid. This powder may be of commercial origin or may be prepared in accordance with the teaching of the patents already mentioned, especially U.S. Pat. No. 5,436,135. The solution having a volume of 1.5 litre is adjusted to a collagen concentration of 3 g/l. It is filtered under sterile conditions through a membrane of cellulose acetate having a porosity of from 0.22 to 0.45 micron in order to remove the bacteria and other microbes which may be present in the starting product. The filtered solution is collected in a sterile flask under a hood with sterile laminar flow and will not leave this space before being concentrated and distributed in syringes. All of the necessary equipment is sterilised by previous irradiation at 30kGray. The filtered acidic solution is then heated to a temperature of from 20 to 25° C., and then disodium phosphate Na₂HPO₄, 12H₂O, is added in order to reach a final concentration of 20 mM, which brings about a precipitation of the collagen in colloidal form and then fibrillar form. The precipitation is completed by adjusting the pH to 7.2 with NaOH 1N sodium hydroxide.

The dilute suspension of collagen fibrils is then introduced by gravity into a reservoir funnel connected to the tube of filtering cloth, which is itself closed at the other end by a clip. The filtering cloth marketed by the company organised under Swiss law, Sefar Fyltis, located at Freibach CH 9425 Thal, under the reference Nycom PA 1 42072 AC, 03-1/1, having a mesh aperture of 1 micron, is excellently suited to this application. This cloth can be produced in the form of a continuous tube which has an inside diameter of from 10 to 60 mm and the sides of which are fused by ultrasound. The reference of the selected product is a Sefar Nitex 03-1/1 tube having a diameter of 20 mm. For a diameter of filtering tube of 20 mm and a length of 60 cm, it is possible to concentrate the 1.5-litre collagen suspension up to approximately 20-fold overnight. The next morning the reservoir of collagen to be filtered is empty. The final concentration in the filtering tube can then be completed by surrounding the tube with a roll of cellulose filter paper. It is thus possible to prepare a fibrillar gel without air bubbles at a concentration of from 3 to 6%. Since the fibrillar gel of sterile collagen is contained in a cylindrical tube, its transfer to a sterile receptacle can be carried out by simple manual pressure on the supple walls of the tube. The sterile distribution of the collagen gel in 1 ml syringes, by an automatic machine, under sterile flow, is thus facilitated.

An increase in scale is readily brought about by adding several identical filtering systems, side by side, to the sterile container. These systems can be connected to the same reservoir containing several tens of litres of the suspension to be concentrated.

The major advantage of this process is that it requires only a minimum of manual operations and can be carried out exclusively in the sterile container. The final level of sterility obtained is therefore optimum.

### EXAMPLE 2

### Concentration of a Suspension of Acidic Collagen Precipitate

Some collagens, such as type IV collagen, do not form a fibrillar precipitate in the presence of phosphate at a neutral pH. For this collagen, as for all the other types, whether they are fibrillar or not, acidic precipitation by the addition of NaCl at a final concentration of from 40 to 50 g/l is possible.

The acidic precipitate of any one of the types of collagen can then be harvested by the same operating mode as in Example 1. If the initial acidic solution has been filtered under sterile conditions through membranes having a porosity of 0.22 or 0.45 micron, a sterile concentrated precipitate is finally obtained. This concentrated acidic precipitate is contained in the filtering tube which can then be closed at its upper portion, as at its lower portion, and subsequently immersed in a physiological solution having a neutral pH if necessary, in order to correct the pH thereof slowly. The major advantage compared with a dialysis tube is that the wall of this filtering tube allows the water to pass through by filtration (and not by very slow osmosis) and therefore there is no risk of it bursting. For the same reason, the osmotic effect of dilution is largely avoided and the preparation of acidic or neutral concentrates of all the collagen types is thus possible.

These concentrates can be used for the manufacture of known collagen biomaterials, gels, films, sponges, granules, optical lenses, impregnated materials, in accordance with known and published techniques.

1. Process for the preparation of sterile collagen, especially injectable collagen, comprising the steps of preparing a purified acidic collagen solution, sterilizing that solution, especially by filtration through a filter or a sterilizing membrane, precipitating the sterile solution in the form of a suspension, and concentrating the collagen suspension, in which process the suspension is placed in an elongate supple tubular filter constituting a porous filtering wall, permeable to the liquid but not permeable to the collagen fibers, and is concentrated by the slow passage of the liquid through the wall, without any pressure other than gravity, in order to prevent the filtering wall from becoming clogged by the collagen fibers.

2. Process according to claim 1, wherein the liquid is absorbed by a previously sterilized absorbent layer applied to the wall, or forming part of that wall, and is released outside the absorbent layer.

3. Process according to claim 2, wherein this absorption is effected towards the end of the concentration process.

4. Process according to claim 1, wherein the steps are carried out in the same sterile environment, especially in a sterile hood.

5. Process according to claim 4, wherein the preliminary steps of obtaining purified collagen and/or the subsequent steps of distributing the concentrated collagen in sterile syringes or receptacles are also carried out in that same sterile environment.

6. Process according to claim 1, wherein the tubular filter is formed by a textile sheet.

7. Process according to claim 1, wherein the filter has a porosity of the order of 1 micron.

8. Process according to claim 2, wherein the absorbent layer is selected from cellulose, hydrophilic polyesters and other hydrophilic polymers.

9. Process according to claim 1, wherein a means for receiving the liquid flowing from the remote face of the absorbent layer, and/or a device for evaporating the liquid is (are) provided.

10. Concentrated sterile preparations of collagen, especially of injectable collagen, especially for filling tissue or cutaneous wrinkles, which preparations are obtained by the process according to claim 1.
